(19) United States
(12) Patent Application Publication
(10) Pub. No.: US 2011/0262325 A1
(43) Pub. Date: Oct. 27, 2011

(54) HAZARDOUS SUBSTANCE REMOVING MATERIAL AND A METHOD FOR REMOVING HAZARDOUS SUBSTANCE

(75) Inventor: Hiroshi Iwanaga, Kanagawa (JP)

(73) Assignee: FUJIFILM Corporation, Minato-ku, Tokyo (JP)

(21) Appl. No.: 12/675,044

(22) PCT Filed: Sep. 18, 2008
(86) PCT No.: PCT/JP2008/067357

§ 371 Date: Feb. 24, 2010

(30) Foreign Application Priority Data
Sep. 19, 2007 (JP) 2007-241838

(51) Int. Cl. B01D 53/85; C08G 71/04; C08F 20/00; A61L 2/23; B01D 15/38; B01D 39/16; A61L 9/00; C07K 16/00; C08F 16/06

(52) U.S. Cl. 423/210; 530/391.1; 525/54.1; 210/764

(57) ABSTRACT

An object to be solved by the present invention is to provide a highly reliable hazardous substance removing material capable of efficiently capturing and quickly inactivating hazardous substances of microbe origin such as viruses and bacteria, so as to minimize influences on the human body, such material being unlikely to be influenced by, for example, dimensional changes derived from dynamical and physical properties or a use environment (particularly humidity). The present invention provides a hazardous substance removing material which comprises a support having antibodies supported thereon, wherein a support is composed of a fiber which comprises at least one polymer having a carbonyl group and/or an ether group and has a volume swelling degree of not less than 1.1% to less than 10% in a water at 20° C.

### TECHNICAL FIELD

The present invention relates to a hazardous substance removing material comprising a fiber provided with antibodies, and a method for removing hazardous substance using the same.

### BACKGROUND ART

In recent years, infectious diseases caused by bacteria, molds, viruses, and the like have been recognized as social problems. For instance, there is a concern of mass infection in general public places such as hospitals and public facilities. Particularly in the case of hospital infection, the misuse of antibiotics and the like causes the generation of MRSA (Methicillin-resistant Staphylococcus aureus), for example.

In view of the above, recent buildings are provided with a duct in each room in such a manner that air is circulated through the duct, using an air-conditioner, so as to control the room temperature or other conditions of the whole building. Thus, bacteria, molds, viruses, and the like floating in a facility are often diffused into the entire facility through such an air conditioner. Therefore, it is considered that blocking of a route for air-mediated infection is particularly effective. Specifically, a finely woven filter is provided to an air distribution part of an air conditioner, air purifier, or the like such that bacteria, mold, viruses, or media therefore such as floating fine objects (e.g., dusts) in air are allowed to be adsorbed to such filter. Alternatively, titanium oxide or a strongly acidic sterilizing zone is provided to the same such that bacteria, molds, and viruses passing therethrough are inactivated and removed.

However, upon such removal by adsorption, if a hazardous substance is a bacterium, virus, or the like, bacteria having captured with a filter might be detached therefrom so as to be reactivated and affect human bodies. In addition, in the case of a method for inactivating a hazardous substance by allowing the hazardous substance to pass through titanium oxide or a strongly acidic sterilizing zone so as to inactivate the hazardous substance, inactivation is time-consuming to a certain extent and the effects obtained thereby are not always sufficient, which has been problematic.

In order to solve the above problem, a method wherein an antigen-antibody reaction is used to inactivate a hazardous substance has been suggested. For example, a method using a relatively inexpensive chicken egg antibody instead of a conventionally used expensive monoclonal antibody or the like (e.g., JP Patent No. 3840978) and, as a support, a fiber with an official moisture regain of 7% or more has been disclosed (JP Patent No. 3642340, and Daikin Industries, Ltd., the air purifier product catalog, pp. 06-11 and 14).

In order to maintain the activity of antibodies, the humidity environment in the vicinity of the fiber has to be indispensably controlled, and a high hygroscopic material such as a cellulosic fiber is used therein. However, in practice, if the content of the cellulosic fiber is high, the fiber itself becomes fragile and tends to be broken due to external influences generated in a filter processing step or upon used of the filter. Needless to say, breakage of a base material for a filter results in leakage of an object to be removed, leading to deficient filter functions. Further, a fiber having high hydrophilicity tends to experience volume expansion due to moisture absorption and subsequent deformation. Thus, structure destruction and changes in filter diameter are caused thereby. As a result, removal efficiency and performance reliability deteriorate, which are problematic.

Under the above circumstances, there has been a demand for the development of novel fiber materials having appropriate hydrophilicity for maintaining antibody activity and being unlikely to be influenced by, for example, dimensional changes derived from dynamical and physical properties or an environment (particularly humidity), whereby the above problems can be solved.

### DISCLOSURE OF THE INVENTION

An object to be solved by the present invention is to solve the problems of conventional hazardous substance removing materials. That is to say, an object to be solved by the present invention is to provide a highly reliable hazardous substance removing material capable of efficiently capturing and quickly inactivating hazardous substances of microbe origin such as viruses and bacteria, so as to minimize influences on the human body, such material being unlikely to be influenced by, for example, dimensional changes derived from dynamical and physical properties or a use environment (particularly humidity). Moreover, another object to be solved by the present invention is to provide an efficient method for removing hazardous substances using the hazardous substance removing material.

In order to solve the above objects the inventors of the present invention have conducted intensive studies. As a result, they have found that a hazardous substance removing material capable of efficiently capturing and quickly inactivating hazardous substances, so as to minimize influences on the human body, being unlikely to be influenced by, for example, dimensional changes derived from dynamical and physical properties or a use environment (particularly humidity), and having high reliability can be provided by having antibodies supported on a support which is composed of a fiber which comprises at least one polymer having a carbonyl group and/or an ether group and has a volume swelling degree of not less than 1.1% to less than 10% in a water at 20° C. This has led to the completion of the present invention.

Thus, the present invention provides a hazardous substance removing material which comprises a support having antibodies supported thereon, wherein a support is composed of a fiber which comprises at least one polymer having a carbonyl group and/or an ether group and has a volume swelling degree of not less than 1.1% to less than 10% in a water at 20° C.

Preferably, the polymer is cellulose ester, vinylon, acrylic, or polyurethane.

Preferably, the cellulose ester is cellulose acylate.

Preferably, the polymer is a polyamide.

Preferably, the polyamide is nylon 6, nylon 66, or polyacrylamide.

Preferably, the official moisture regain of a fiber constituting the support is not less than 1% to less than 7%.

Preferably, the fiber constituting the support has a three-dimensional structure of a hole or a projection having an average diameter of not less than 50 nm to not more than 1 μm on the surface thereof.

Preferably, the fiber constituting the support has a tensile elastic modulus in a dried state of 25% or more.

Preferably, the fibers constituting the support partially adhere to each other so as to form a three-dimensional network.

Preferably, the fiber diameter of the fibers constituting the support is 100 nm or less.

Preferably, the antibody is a chicken egg antibody.

Another aspect of the present invention provides a method for removing hazardous substance, which comprises removing a hazardous substance in a gas phase or liquid phase using the aforementioned hazardous substance removing material of the present invention.

### BRIEF DESCRIPTION OF THE DRAWINGS

FIG. 1 shows a spinning apparatus used in Examples 1 to 4 and Comparative Example 1.

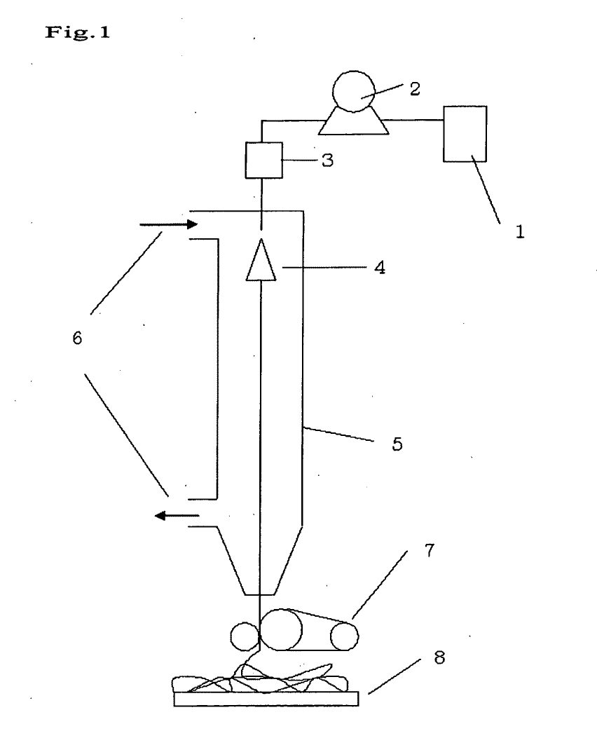

FIG. 2 shows an electrospinning apparatus used in Examples 5 and 6.

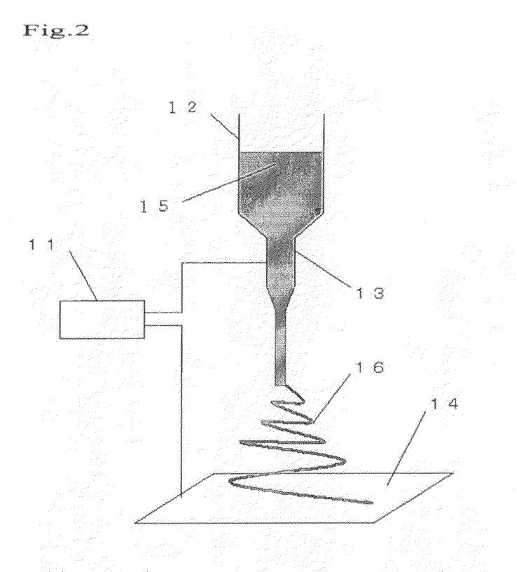

1 denotes Polymer solution; 2 denotes Gear pump; 3 denotes Filter; 4 denotes Nozzle; 5 denotes Spinning cylinder; 6 denotes air; 7 denotes Taking-over roller; 8 denotes Table; 11 denotes Power-supply unit; 12 denotes Syringe; 13 denotes Needle; 14 denotes Collector; 15 denotes Polymer solution; and 16 denotes Nanofiber

### BEST MODE FOR CARRYING OUT THE INVENTION

Hereinafter, the present invention is described in more detail.

The hazardous substance removing material of the present invention is a hazardous substance removing material which comprises a support having antibodies supported thereon, wherein the support is composed of a fiber comprising a polymer containing, as a main component, at least one selected from the group consisting of cellulose ester, polyamide, vinylon, acrylic, and polyurethane, such fiber having a volume swelling degree of not less than 1.1% to less than 10% in a water at 20° C. Thus, such material has sufficient mechanical strength and is unlikely to be influenced by, for example, dimensional changes derived from a use environment (particularly humidity). In addition, it can preserve moisture to maintain the activity of antibodies to be supported. Therefore, an antigen-antibody reaction can be stably used for removing a hazardous substance in a gas phase. In addition, an antibody captures a specific hazardous substance and thus it is possible to remove hazardous substance in an accurate manner by selecting the relevant antibody specific to a hazardous substance. Further, an antibody itself has functions to sterilize/inactivate some types of hazardous substances. In such cases, there is no need to combine techniques for sterilization/inactivation of a hazardous substance. Further, the hazardous substance removing material alone can remove a hazardous substance.

Preferably, a main material which forms a support is a fiber comprising, as a main component, at least one selected from the group consisting of cellulose ester, vinylon, acrylic, and polyurethane. In addition, as a main material which forms a support, a fiber comprising, as a main component, polyamide is also preferable. According to the present invention, the term “main component” means a component that accounts for 25% or more in terms of mass fraction with respect to the total mass of fibers.

According to the present invention, the term “cellulose ester” refers to a cellulose derivative obtained by esterifying a hydroxyl group of cellulose with an organic acid. Examples of an organic acid used for esterification include fatty carboxylic acids such as acetic acid, propionic acid, and butyric acid and aromatic carboxylic acids such as benzoic acid and salicylic acid. They may be used alone or in combination. The rate of substitution of a hydroxyl group of cellulose with an ester group is not particularly limited; however, it is preferably 60% or more.

According to the present invention, a cellulose acylate fiber is preferable among the group of main materials which form a support. The term “cellulose acylate” used herein refers to cellulose ester in which some or all of hydrogen atoms of a hydroxyl group of cellulose are substituted with an acyl group. Examples of an acyl group include an acetyl group, a propionyl group, and a butylyl group. In terms of structure, a single group among the above examples may be substituted, or two or more acyl groups may be subjected to mixed substitution. The total sum of degrees of acyl group substitution is preferably 2.0 to 3.0, more preferably 2.1 to 2.8, and particularly preferably 2.2 to 2.7. Among them, cellulose acetate, cellulose acetate propionate, or cellulose acetate butylate capable of achieving such degree of substitution is preferable, and cellulose acetate is most preferable. In general, it has been known that a solvent for cellulose acylate varies depending on the degree of esterification. It is also possible to produce a support with cellulose acylate having a high esterification rate in advance and then subject the support to alkali hydrolysis treatment or the like for hydrophilicization of the surface thereof.

It is possible to form a sufficiently practical hazardous substance removing material consisting of a cellulose acylate fiber. However, in order to further improve strength, dimensional stability, and the like, a support may be formed with a mixed fiber (e.g., polyester-based fiber/polyolefin-based fiber/polyamide-based fiber/acrylic-based fiber). When a mixed fiber is used, the mass fraction of a cellulose acylate fiber is preferably 50% or more and more preferably 70% or more.

According to the present invention, a polyamid fiber is preferable among the group of main materials which constitutes a support.

According to the present invention, the term “polyamide” refers to a fiber comprising a linear polymer having a chemical structure unit comprising an amide bond.

Among polyamides, a linear aliphatic polyamide, which is a combination of an aliphatic diamine such as ethylenediamine, 1-methylethylenediamine, 1,3-propylenediamine, or hexamethylenediamine and an aliphatic dicarboxylic acid such as malonic acid, succinic acid, or adipic acid, is preferable. Nylon 66 is particularly preferable.

In addition to the above diamine and dicarboxylic acid, aliphatic polyamide comprising a single component or copolymer components selected from among the following examples can be used: lactams such as ε-caprolactam and laurolactam; aminocarboxylic acids such as aminocaproic acid and aminoundecanoicacid; and para-aminomethyl benzoic acid. Nylon 6 produced using ε-caprolactam alone is particularly preferable.

In addition to the above, the following may be used: an aliphatic polyamide in which cycloaliphatic diamine such as cyclohexanediamine, 1,3-bis(aminomethyl)cyclohexane, or 1,4-bis(aminomethyl)cyclohexane is partially or entirely used as a material aliphatic diamine; and/or an aliphatic polyamide in which cycloaliphatic dicarboxylic acid such as 1,4-cyclohexane dicarboxylic acid, hexahydroterephthalic acid, or hexahydroisophthalic acid is partially or entirely used as dicarboxylic acid.

Further, examples of the above polyamide further include a polyamide with decreased water absorbability and an improved elastic modulus in which aromatic diamine such as aliphatic paraxylylene diamine (PXDA) or metaxylylene diamine (MXDA) and aromatic dicarboxylic acid such as terephthalic acid are partially used as starting materials. Moreover, a polymer having a side chain comprising an amide bond such as polyacrylic acid amide, poly(N-methylacrylic acid amide), or poly(N,N-dimethylacrylic acid amide) may be used.

Among polyamides, nylon 66 or nylon 6 is most preferable. This is because the following properties of such polyamide are preferable to be used as the support of the present invention: appropriate hygroscopic properties derived from amide bonds; ease of inducing fiber axis orientation of a molecular chain comprising a long-chain fatty acid having an appropriate length that results in relatively high extensibility; a dynamic and kinetic tendency to not be melted due to high melting temperature and thermal capacity (resistance to melting); flexibility of a molecular chain comprising a long-chain fatty acid; and a tendency to not cause fibrillation or kink band formation (such tendency being imparted as a result of formation of a hydrogen bond between amide bonds), that is to say, repetitive bending and stretching properties.

Preferably, a polyamide in which an amide bond in a chemical structure unit exists on a side chain but not on a main chain can be used. Examples thereof include polyacrylamides such as poly(N-isopropylacrylamide), poly(N,N-dimethylacrylamide), and poly(N-hexylacrylamide). In general, a polymer having a side chain comprising an amide bond has high hydrophilicity and thus tends to be swollen/deformed. Thus, it is preferable that a physically crosslinked polymer be formed with the use of a gelatinization phenomenon or a polymer be hydrophobized by a method comprising introducing an alkyl group, for example.

Likewise, in order to improve strength or dimensional stability, a support may be reinforced with other appropriate structural materials such as metals, high-molecular materials, and ceramics. It is desirable that such reinforcing materials be used for a part which is not positioned on the substantially outermost surface of a face to which a hazardous substance removing material is applied (such material being used for, for example, the face located opposite to such face or a core material).

According to the present invention, the term “vinylon” refers to a fiber comprising a linear polymer containing vinyl alcohol units (65% by mass or more) and having a moisture regain of less than 7% obtained at least 1 week after placement of such fiber in an environment at a temperature of 20° C. and at a humidity of 65%. Such fiber may be obtained by formalizing a hydroxyl group of vinyl alcohol. Also, it may be a polymer obtained by subjecting a hydroxyl group to boric acid crosslinking or a non-formalized fiber subjected to a waterproof treatment by a known method such as an alkaline spinning method or a cooled gel spinning method. The above fiber may contain, as non-vinyl-alcohol-unit component, an ethylene chain or a vinyl acetate chain. However, it is preferably a fiber formed with a vinyl alcohol support. Further, it is most preferably a non-formalized fiber obtained by cooled gel spinning. This is because a non-formalized fiber has uniform properties and high degree of orientation/crystallization and thus excellent mechanical properties and reliability can be obtained.

In general, vinylon is superior to other fibers in terms of high strength, high elastic modulus, appropriate hydrophilicity, weather resistance, chemical resistance, adhesiveness, and the like. Thus, the preferable properties thereof can be used for the support of the present invention support.

According to the present invention, the term “acrylic” refers to a fiber comprising recurring units of an acrylonitrile group (mass percentage: 40% or more). Examples thereof include a homopolymer of acrylonitrile; a copolymer of acrylnitrile and a nonionic monomer such as acrylic ester, methacrylic ester, or vinyl acetate; a copolymer of acrylonitrile and an anionic monomer such as vinylbenzenesulfonate or allylsulfonate; and a copolymer of acrylonitrile and a cationic monomer such as vinylpyridine or methylvinylpyridine.

In general, an acrylic fiber is produced by an organic solvent wet spinning method. In this method, when a spinning stock solution is formed into a coagulated thread in a coagulating bath, water serving as a coagulant is mixed with the spinning stock solution that is spinning-twisted from a nozzle and a spinning solvent is externally diffused from the spinning-twisted stock solution. At such time, water and an organic solvent (e.g., DMF or DMAc) are mutually diffused such that a polymer deposits, resulting in the formation of a line of coagulated thread having a structure in which many cavities are connected to each other in a net form. In addition, such thread is characterized by deformation of a fiber section caused by volume contraction as a result of diffusion of a solvent into a coagulating bath during coagulation and by formation of concave-convex portions as a result of macrofibril structure formation on the surface thereof. Such fine structure is preferable as a structure of a support used in the present invention in terms of an increase in specific surface area or the ease of antibody loading.

An acrylic fiber used in the present invention varies depending on the composition of a starting material polymer, a spinning method, post-treatment conditions during production, and the like. However, in general, a bulky fiber having appropriate hydrophilicity and high weather resistance can be obtained, which is advantageous.

The term “polyurethane” used in the present invention refers to a fiber comprising a linear synthetic polymer in which bonds between monomers or basic substrate polymer units are mainly urethane bonds. Preferably such fiber contains a polyurethane segment at a mass percentage of 85% or more. Preferably, such polyurethane is a block copolymer of segmented polyurethane comprising a soft segment that is soft and have a molecular weight of several thousands and a low melting point and a hard segment that is rigid and have high cohesion and a high melting point. For a soft segment, polyether such as polypropylene glycol or polytetramethylene glycol can be used. For a hard segment, a urethane group formed with 4,4′-diphenylmethane diisocyanate, m-xylene diisocyanate, or the like can be used. Polyurethane is generally characterized by a high elasticity. Also, it is further characterized by good extensibility, high restoring force upon expansion and contraction, antidegradation properties better than those of rubber materials, formation into thin fibers, and the like, although the characteristics thereof vary depending upon differences in terms of a primary structure of a high-molecular chain such as the distribution and chemical structure of each segment and upon differences in terms of a secondary structure derived from different spinning conditions. Thus, when polyurethane is used as a support of the present invention, such characteristics can be utilized.

Further, the present inventors have found that one requirement of the present invention is a tendency to not be influenced by dimensional changes derived from moisture. Specifically, the volume swelling degree in pure water at 20° C. of a fiber constituting the support used in the present invention is not less than 1.1% to less than 10%, preferably not less than 1.1% to less than 8%, particularly preferably not less than 1.1% to less than 6%, and most preferably not less than 1.1% to less than 5%. In addition, the volume swelling degree in pure water at 20° C. of a fiber according to the present invention refers to the volume swelling degree which is obtained by measuring the fiber density of each sample in a dried state before immersion of the sample in pure water at 20° C. for 1 hour and that after such immersion. Such values are obtained by the density gradient tube method (JIS-K7112).

Regarding mechanical and physical properties and dimensional stability of a fiber constituting a support, the tensile elastic modulus in a dried state is preferably 25% or more. The term “tensile elastic modulus in a dried state” used herein refers to the degree of elongation at break of a fiber in a tensile test at 20° C., provided that such fiber has been dried for a sufficiently long period of time. In general, a fiber having a tensile elastic modulus in a dried state of 10% or more is preferable for processing such as fabric formation. In order to prevent breakage upon filter processing or practical use (such breakage leading to reduction in filtration efficiency), the tensile elastic modulus is preferably 25% or more, more preferably 30% or more, and most preferably 35% or more.

The official moisture regain of the fiber constituting the support is preferably not less than 1.0% to less than 7%, more preferably not less than 3.0% to less than 6.5%, most preferably not less than 5.0% to less than 6.5%. Within the above range of official moisture regain, the expression of the activity of a supported antibody and the mechanical strength, rigidity, dimensional change stability in a use environment (particularly humidity) of a support can be achieved. Further, a filter obtained therewith can exhibit high performance and reliability.

In addition, the term “moisture regain” used herein refers to an official moisture regain. The term “official moisture regain” refers to a moisture regain of a fiber that has been left in an environment at 20° C. and at a relative humidity of 65% for long period of time. Moreover, when a fiber is a mixed fiber further containing a different fiber, the term refers to the official moisture regain of the total mixed fibers.

Preferably, the surface of a fiber constituting a support has fine concave-convex portions several tens nanometers to several micrometers in size. The shape of a concave-convex portion may be a three-dimensionally shaped groove or ridge which is formed in the direction parallel to the fiber direction or in the direction vertical to the same, that is to say, in a concentric direction with respect to the fiber axis. Such three-dimensionally shaped groove or ridge may exist at an arbitrary proportion or density, provided that an arbitrary angle is formed between such groove or ridge and a line extending in the direction parallel thereto, in the direction vertical thereto, or in the direction between such parallel direction and such vertical direction. A sample obtained by a known method for cellulose acetate fiber spinning is known to have a fiber section having a variable chrysanthemum-like shape as a result of skin layer formation on the surface thereof and depression of a skin layer due to solvent drying. In a preferred embodiment, such concave-convex portions are used for the present invention.

The above fine concave-convex portions several tens nanometers to several micrometers in size may have holes and/or projections. Preferably, such holes or projections have an average diameter of 50 nm to 1 μm. Such holes and projections can be formed by, for example, cavitation of a solution or they can be formed in a spinning step of a method using a solution in which a fine dispersoid is dispersed (e.g., a mixture containing a slurry in which barium sulfate particles are dispersed) or in a subsequent step by a method involving hydrolysis of an acyl group, surface oxidation treatment, or the like (e.g., the exposure of a cellulose portion on the fiber surface with the use of an alkaline water solution followed by generation of microcraters by an enzyme treatment).

The average fiber diameter of a fiber used for the hazardous substance removing material of the present invention is preferably 50 μm or less, more preferably 10 μm or less, particularly preferably 1 μm or less, and most preferably 100 nm or less. The average fiber diameter of the present invention is obtained by measuring the diameters of fibers in arbitrarily selected 300 sites on a scanning electron microscope (SEM) image for observation and averaging the results by calculation.

As to the method for producing the fiber used in the present invention, there are typical production methods such as melting spinning, wet spinning, dry spinning, and dry-wet spinning, and methods in which the fiber is made fine by a physical process (such as strong mechanical shearing using an ultrahigh pressure homogenizer), although dry spinning or dry-wet spinning is preferably employed for obtaining a stable quarity of product. For producing an uniform fiber having an average fiber diameter of 100 nm or less, the electrospinning method disclosed in “Kakou Gijyutsu (Processing Technology)”, 2005, Vol. 40, No. 2, p. 101 and p. 167; “Polymer International”, 1995, Vol. 36, pp. 195-201; “Polymer Preprints”, 2000, Vol. 41(2), p. 1193; “Journal of Macromolecular Science: Physics”, 1997, B36, p. 169; and the like is preferably used.

Regarding the solvent used for the spinning, any solvent may be used as long as it dissolves the resin used for synthetic resin fibers. Examples thereof include: chloride-based solvents such as methylene chloride, chloroform, and dichloroethane; amide-based solvents such as dimethylformamide, dimethylacetamide, and N-methylpyrrolidone; ketone-based solvents such as acetone, ethyl methyl ketone, methyl isopropyl ketone, and cyclohexanone; ether-based solvents such as THF and diethyl ether; and alcohol-based solvents such as methanol, ethanol, and isopropanol. These solvents may be used either singularly, or in mixtures of a plurality of types thereof.

The resin solution used for the electrospinning method may be added with a salt such as lithium chloride, lithium bromide, potassium chloride, and sodium chloride.

Preferably, fibers constituting a support of the hazardous substance removing material of the present invention partially adhere to each other such that a structure forming a three-dimensional network is obtained. The use of such structure results in the improvement of mechanical tolerance upon processing or practical use, leading to the improvement of reliability of the hazardous substance removing material. Further, antibody-supporting properties of the present invention can be improved. Adhesion between fibers can be observed by a method involving SEM or the like. The density of fiber adhesion points is preferably 10 adhesion points or more in a 1-mm square on the projected surface area of the hazardous substance removing material and preferably 100 adhesion points or more in the same.

Regarding a method for forming adhesion points, adhesion points may be formed by a dry spinning method or by a melt spinning method. After spinning, adhesion point formation treatment may be carried out by heating or adding an adhesive/plasticizing solvent or the like. In view of production cost, it is preferable to form adhesion points by a dry spinning method with the use of an appropriate solution formulation.

The antibody used for the hazardous substance removing material of the present invention is a protein which is reactive (antigen-antibody reaction) specifically to a specific hazardous substance (antigen), has a molecule size of 7 to 8 nm, and is in a Y-shaped molecular form. A pair of branch portions of the antibody in the Y-shaped molecular form are called Fab, and a stem portion thereof is called Fc, among which the Fab portions capture the hazardous substance.

The type of the antibody corresponds to the type of the hazardous substance to be captured. Examples of the hazardous substance to be captured by the antibody include bacteria, fungi, viruses, allergens, and mycoplasmas. Specifically, the bacteria include, for example: the genus Staphylococcus (such as Staphylococcus aureus and Staphylococcus epidermidis), Micrococcus, Bacillus anthracis, Bacillus cereus, Bacillus subtilis, and Propionibacterium acnes, as gram-positive bacteria; and Pseudomonas aeruginosa, Serratia marcescens, Burkholderia cepacia, Streptococcus pneumoniae, Legionella pneumophilia, and Mycobacterium tuberculosis, as gram-negative bacteria. The fungi include, for example, Aspergillus, Penicillius, and Cladosporium. The viruses include influenza viruses, coronavirus (SARS virus), adenovirus, and rhinovirus. The allergens include pollens, mite allergens, and cat allergens.

Examples of methods for producing the antibody include: a method in which an antigen is administered to an animal such as a goat, a horse, a sheep, and a rabbit, and a polyclonal antibody is refined from the blood thereof; a method in which spleen cells of an animal to which an antigen is administered and cultured cancer cells are subjected to cell fusion and a monoclonal antibody is refined from a culture medium thereof or from a humor (such as ascites) of an animal in which the fussed cells are implanted; a method in which an antibody is refined from a culture medium of genetically modified bacteria, plant cells, or animal cells to which antibody producing gene is introduced; and a method in which a chicken to which an antigen is administered is allowed to lay an immune egg and a chicken antibody is refined from yolk powder obtained by sterilizing and spray-drying the yolk of the immune egg. Of all the above methods, the method for obtaining the antibody from a chicken egg enables easy mass production of the antibody, reducing the cost of the hazardous substance removing material.

The antibody used for the hazardous substance removing material of the present invention is preferably a chicken egg antibody.

It is desirable that the support constituting the hazardous substance removing material of the present invention is subjected to antibacterial treatment such as coating of an agent containing an antibacterial agent and/or antifungal treatment such as coating of an agent containing an antifungal agent. The antibody is principally a protein, and particularly, the chicken egg antibody is food, and the antibody may also accompany a protein other than the antibody. These proteins might serve as food for bacteria and fungi to proliferate. However, if the support is subjected to antibacterial and/or antifungal treatment, such multiplication of bacteria and the fungi is suppressed, so that a long-term storage becomes possible.

The antibacterial/antifungal agents include organic silicon quaternary ammonium salts, organic quaternary ammonium salts, biguanides, polyphenols, chitosan, silver-support colloidal silica, zeolite-support silvers, and the like. As to the treatment method, there are a post-treatment method in which an antibacterial/antifungal agent is immersed in or applied to the support made of a fiber, a raw thread/raw cotton improving method in which an antibacterial/antifungal agent is mixed in the step of synthesizing a fiber constituting the support, and the like.

Regarding methods for immobilizing the antibody to the support, there are: a method in which, after a support is subjected to silane treatment using γ-aminopropyl-triethoxysilane or the like, an aldehyde group is introduced on the surface of the support by glutaraldehyde or the like, to effect a covalent bond between the aldehyde group and an antibody; a method in which an untreated support is immersed into an aqueous solution of an antibody to cause ion boding, thereby immobilizing the antibody to the support; a method in which an aldehyde group is introduced to a support having a specific functional group to effect a covalent bond between the aldehyde group and an antibody; a method in which a support having a specific functional group is ion-bonded to an antibody; and a method in which a support is coated with a polymer having a specific functional group, followed by an introduction of an aldehyde group to effect a covalent bond between the aldehyde group and an antibody.

Here, the specific functional group includes an NHR group (R represents an alkyl group of any of methyl, ethyl, propyl, and butyl except H), an NH₂ group, a C₆H₅NH₂ group, a CHO group, a COOH group, and an OH group.

Moreover, there is also a method in which a functional group on the surface of the support is replaced with another functional group using BMPA (N-β-Meleimidopropionic acid) or the like, to effect a covalent bond between the resultant functional group and an antibody (an SH group is replaced with a COOH group by BMPA).

Further, there is another method in which a molecule (such as an Fc receptor and a protein A/G) which is selectively bindable to the Fc portion of the antibody is introduced on the surface of a support, to cause it to be bonded to the Fc of the antibody. In this case, the Fab for capturing a hazardous substance are arranged outwards from the support to cause increase in contact possibility of the hazardous substance to the Fab, resulting in efficient capturing of the hazardous substance.

The antibody may be supported on the support through a linker. In this case, the degree of freedom of the antibody on the support increases, so that the antibody can readily reach the hazardous substance, attaining a high removal performance. Divalent or multivalent crosslinking reagent may be used as the linker. Specifically, there are listed maleimide, NHS (N-Hydroxysuccinimidyl) ester, imide ester, EDC (1-Ethyl-3-[3-dimethylaminopropyl]carbodiimido), and PMPI (N-[p-Maleimidophenyl]isocyanete), which are selectively or non-selectively bonded to a target functional group (an SH group, an NH₂ group, a COOH group, and an OH group). Moreover, crosslinking agents have different crosslinking distances (spacer arm), and therefore, the distance can be selected within the range between about 0.1 nm to about 3.5 nm according to the target antibody. In view of efficient capturing of the hazardous substance, the linker is preferably bindable to the Fc of the antibody.

Regarding the method for introducing the linker, either one of the following methods may be possible: a method in which a linker is bonded to an antibody, and then the resultant linker is further bonded to the antibody; and a method in which a linker is bonded to a support, and then an antibody is bonded to the linker on the support.

The hazardous substance removing material of the present invention can be used for a filter for an air purifier, a mask, a wipe sheet, and the like.

When the hazardous substance removing material of the present invention is used for an air purifier filter, it may be used in combination with the following conventional filters and any other conventional filters: a prefilter for removing dusts, a dust removal filter, a photocatalyst filter having deodorant effects, an antibacterial filter for removing other hazardous substances, and a VOC-absorbing filter.

### EXAMPLES

The features of the present invention are hereafter more specifically described with reference to examples and comparative examples. Materials, their quantities consumed, proportions thereof, contents of processing, processing procedures, and the like set forth in the following examples can be appropriately modified without departing from the sprit of the present invention. Accordingly, the scope of the present invention is not to be construed as being limited to the specific examples shown below.

### Example 1

An acetone-water (97:3) solution (25% by mass) containing cellulose acetate

(total degree of substitution: 2.4; number average molecular weight: 30,000, manufactured by Aldrich) was heated to 60° C. and then was squirted with air out of a nozzle of 0.1 mm in diameter at a spinning rate of 500 m/m for nonwoven fabric formation. Accordingly, a nonwoven fabric N-1 with a membrane thickness of 85 μm was obtained. A spinning cylinder was heated to 100° C. with a heater. The average fiber diameter was measured by SEM and found to be 8 μm.

### Example 2

A nonwoven fabric N-2 was obtained in the same manner as that of Example 1 except that cellulose acetate propionate (degree of propionate substitution: 2.1; degree of acetate substitution: 0.2; number average molecular weight: 25,000, manufactured by Aldrich) was used. The average fiber diameter was measured by SEM and found to be 12 μm.

### Example 3

A nonwoven fabric N-3 was obtained in the same manner as that of Example 1 except that the temperature of a spinning cylinder was set at 60° C. and the obtained nonwoven fabric was dried at 100° C. for 1 hour. The average fiber diameter was measured by SEM and found to be 11 μm. Further, it was observed that fibers adhered to each other in a manner such that fiber contact points formed nodes.

### Example 4

A nonwoven fabric N-4 was obtained in the same manner as that of Example 1 except that a mixed solution to which an organosol containing colloidal silica (MEK-ST, 1% by mass, manufactured by Nissan Chemical Industries, Ltd.) had been added was used instead of the acetone solution used in Example 1. The average fiber diameter was measured by SEM and found to be 9 μm. Further, it was observed that silica aggregates with the average size of 200 nm were distributed on the fiber surfaces.

### Example 5

An acetone-water (97:3) solution (10% by mass) containing cellulose acetate (total degree of substitution: 2.4; number average molecular weight: 30,000; manufactured by Aldrich) was used for electrospinning with a nanofiber producing apparatus (manufactured by Kato Tech Co., Ltd.) at a syringe feed rate of 0.05 mm/min with an applied voltage of 15 kV. The resultant was dried in vacuum at 80° C. for 8 hours to thereby prepare a nonwoven fabric N-5 with a membrane thickness 85 μm. The average fiber diameter was measured by SEM and found to be 80 nm.

### Example 6

Nonwoven fabrics N-6 to N-9 (membrane thickness: 85 μm) were prepared in the same manner as that of Example 5 except that the composition of the polymer solution used in Example 5 was changed to the relevant composition shown in table 1. The average fiber diameter was measured by SEM and found to be 90 to 110 nm.

TABLE 1
The compositions of solutions of polymers serving as fiber materials

| Sample No. | Polymer | Solvent composition | Polymer concentration |
|---|---|---|---|
| N-6 | Nylon66 | Formic acid | 5% by mass |
| N-7 | Vinylon | Pyridine | 2% by mass |
| N-8 | Acrylonitrile | DMF | 10% by mass |
| N-9 | Polyurethane | DMF | 5% by mass |

### Comparative Example 1)

A nonwoven fabric H-1 (membrane thickness: 85 μm) was obtained in the same manner as that of Example 1 except that an N-methylmorpholine-N-oxide solution (5% by mass) containing cellulose was used instead of the acetone solution containing cellulose acetate and the sample was dried in vacuum at 80° C. for 8 hours after film forming. The average fiber diameter was measured by SEM and found to be 8 μm.

### Comparative Example 2

A commercially available melt-blown filter (polypropylene, manufactured by Tapyrus Co., Ltd.) was directly used as H-2. The average fiber diameter was measured by SEM and found to be 12 μm.

### (Measurement of Moisture Regain)

The respective samples of N-1 to N-9, H-1, and H-2 were left for 1 week or more in an environment at 20° C. and at a relative humidity of 65%. Then, the moisture regain of each sample was measured using a halogen moisture analyzer MB35 (manufactured by OHAUS).

### (Measurement of Tensile Elastic Modulus in a Dried State)

Sample sections having a size of 1.0 cm×5.0 cm were cut out from the respective samples. The sections were left at 25° C. and at a relative humidity of 5% or less for 1 week in a low-humidity storage cabinet. Then, the tensile elastic modulus was measured at a pulling rate of 3 mm/min at 20° C. using a Tensilon (Tensilon™-25, manufactured by Toyo Baldwin). Then, the tensile elastic modulus in a dried state was obtained based on the degree of breaking elongation (the distance between the chucks was 3 cm). Three samples were measured in one test, and their measured results were averaged to be employed as the tensile elastic modulus in a dried state.

### (Measurement of Swelling Degree)

The volume swelling degree was measured based on the fiber density of each sample in a dried state before immersion of the sample in pure water at 20° C. for 1 hour and that after such immersion. Such values were obtained by the density gradient tube method (JIS-K7112).

### (Antibody Immobilization)

An immune egg delivered by a chicken administered with an antigen was refined to produce influenza virus antibodies (IgY antibodies), and the obtained antibodies were dissolved in a phosphate buffered saline (PBS) to prepare the antibody concentration at 100 ppm. In the prepared solution were immersed the respective samples of N-1 to N-9, H-1, and H-2 at room temperature for 16 to 24 hours, to thereby provide the antibodies onto the surface of the fibers. The obtained samples were left in an environment at 25° C. and 20% RH for 24 hours and then in an environment at 25° C. and 90% RH for 24 hours. This operation was repeated 3 times in each environment (6 times in total).

### (Evaluation of Viral Inactivation Efficiency)

For the test virus solution, a 10-fold dilution of the refined influenza virus with PBS was used. The respective samples N-1 to N-9, H-1, and H-2 were cut into 5 cm square pieces, and were attached and fixed to the center of a virus spray test apparatus. The test virus solution was placed in a nebulizer that had been set on the upstream side, and a virus collecting apparatus was installed on the downstream side. A compressed air was fed from an air compressor to spray the test virus from the nozzle of the nebulizer. A gelatin filter was set on the downstream side from the mask, and an air in the test apparatus was sucked for 5 minutes at a suction flow rate of 10 L/min to collect the virus mist that had passed therethrough.

After the test, the gelatin filter having the captured virus was collected, and the virus infection value after the test solution had passed through the samples was obtained by a TCID 50 (50% tissue culture infective dose) method using MDCK cells. The transient virus removal rate of each sample was calculated by comparing the virus infection values of the gelatin filter with and without the sample.

The results were summarized in Table 2.

TABLE 2
Measured results of physical properties of nonwoven fabrics

| | | Tensile | | Transient | |
|---|---|---|---|---|---|
| | | elastic | | virus | |
| Sample | Moisture | modulus in | Swelling | removal | |
| No. | regain | dried state | degree | rate | Remarks |
| N-1 | 6.3% | 33% | 5.7% | 86% | present invention |
| N-2 | 4.8% | 35% | 5.2% | 81% | present invention |
| N-3 | 6.2% | 34% | 5.4% | 81% | present invention |
| N-4 | 6.4% | 31% | 5.8% | 90% | present invention |
| N-5 | 6.3% | 31% | 5.3% | 99% | present invention |
| N-6 | 4.3% | 41% | 3.1% | 79% | present invention |
| N-7 | 4.8% | 25% | 4.8% | 73% | present invention |
| N-8 | 1.9% | 39% | 1.5% | 71% | present invention |
| N-9 | 1.1% | 630% | 1.2% | 65% | present invention |
| H-1 | 11.2% | 21% | 95.0% | 11% | comparative |
| | | | | | Example |
| H-2 | 0.4% | 38% | 1.0% | No blocking | comparative |
| | | | | | Example |

### Example 7

### Comparison in Terms of Load Dependency

Nonwoven fabrics N-10 to N-11 (membrane thickness: 85 μm) were prepared in the same manner as that of Example 5 except that the composition of the polymer solution used in Example 5 was changed to the relevant composition shown in table 3. The average fiber diameter was measured by SEM and found to be 90 to 110 nm.

TABLE 3
Compositions of solutions of polymers serving as fiber materials

| | | Solvent | Polymer |
|---|---|---|---|
| Sample No. | Polymer | composition | concentration |
| N-10 | Nylon6 | Formic acid | 5% by mass |
| N-11 | Poly(N-isopropylacrylamide) | Distilled water | 2% by mass |

Nest, the IgY-antibody-containing phosphate buffer saline (PBS) solution used above (antibody immobilization) was deposited in a certain amount on N-5, N-6, N-10, N-11, and H-1 by a spray method. Then, the transient removal rate for the virus was evaluated as mentioned above.

TABLE 4
IgY-antibody-load dependency of each sample in terms of viral inactivation
efficiency

| | Load | | | |
|---|---|---|---|---|
| Sample No. | 0.1 mg/m² | 1 mg/m² | 10 mg/m² | Remarks |
| N-5 | 11% | 22% | 99% | present invention |
| N-6 | 81% | 79% | 97% | present invention |
| N-10 | 72% | 79% | 98% | present invention |
| N-11 | 18% | 56% | 95% | present invention |
| H-1 | No blocking | No blocking | 11% | Comparative |
| | | | | Example |

As seen in table 4, in the cases of polyamides (sample Nos. N-6, N-10, and N-11), the virus removal capacity was exhibited with small antibody-supporting amounts. Thus, it was found that antibodies can efficiently be utilized with the use of such polymers.

### INDUSTRIALLY APPLICABILITY

According to the present invention, it becomes possible to produce a hazardous substance removing material capable of removing particles in a gas phase or a liquid phase with a high efficiency, in particular capable of selectively capturing and inactivating hazardous substances of microbe origin such as viruses and bacteria. Moreover, the hazardous substance removing material of the present invention is capable of maintaining the activity of antibodies on the surface of the fiber and keeping a sufficient strength of the fiber itself. Further, the abovementioned hazardous substance removing material is unlikely to be influenced by, for example, dimensional changes derived from a use environment (particularly humidity) and thus it can be used as a highly reliable filter material for air purification and liquid purification. According to the method of the present invention, an air purifier or a liquid purifier capable of efficiently removing hazardous substances in a gas phase or a liquid phase can be produced, which is thus very useful in the industry.

1. A hazardous substance removing material which comprises a support having antibodies supported thereon, wherein a support is composed of a fiber which comprises at least one polymer having a carbonyl group and/or an ether group and has a volume swelling degree of not less than 1.1% to less than 10% in a water at 20° C.

2. The hazardous substance removing material according to claim 1, wherein the polymer is cellulose ester, vinylon, acrylic, or polyurethane.

3. The hazardous substance removing material according to claim 2, wherein the cellulose ester is cellulose acylate.

4. The hazardous substance removing material according to claim 1, wherein the polymer is a polyamide.

5. The hazardous substance removing material according to claim 4, wherein the polyamide is nylon 6, nylon 66, or polyacrylamide.

6. The hazardous substance removing material according to claim 1, wherein the official moisture regain of a fiber constituting the support is not less than 1% to less than 7%.

7. The hazardous substance removing material according to claim 1, wherein the fiber constituting the support has a three-dimensional structure of a hole or a projection having an average diameter of not less than 50 nm to not more than 1 μm on the surface thereof.

8. The hazardous substance removing material according to claim 1, wherein the fiber constituting the support has a tensile elastic modulus in a dried state of 25% or more.

9. The hazardous substance removing material according to claim 1, wherein the fibers constituting the support partially adhere to each other so as to form a three-dimensional network.

10. The hazardous substance removing material according to claim 1, wherein the fiber diameter of the fibers constituting the support is 100 nm or less.

11. The hazardous substance removing material according claim 1, wherein the antibody is a chicken egg antibody.

12. A method for removing hazardous substance, which comprises removing a hazardous substance in a gas phase or liquid phase using the hazardous substance removing material according to claim 1.
